(19) United States
(12) Patent Application Publication
(10) Pub. No.: US 2010/0167335 A1
(43) Pub. Date: Jul. 1, 2010

(54) METHODS FOR DIAGNOSING AND TREATING ALLERGIES

(75) Inventors: Rauno Joks, Port Washington, NY (US); Stephan Kohlhoff, Brooklyn, NY (US)

Correspondence Address: HOFFMANN & BARON, LLP, 6900 JERICHO TURNPIKE, SYOSSET, NY, 11791, US

(73) Assignee: The Research Foundation of State University of New York, Albany, NY (US)

(21) Appl. No.: 12/649,704

(22) Filed: Dec. 30, 2009

Related U.S. Application Data
(60) Provisional application No. 61/141,436, filed on Dec. 30, 2008.

(51) Int. Cl. C12Q 1/02

(52) U.S. Cl. 435/29

(57) ABSTRACT

The present invention relates to methods of screening actives for the treatment of allergic reactions and providing treatment therefor. In particular, the invention relates to the screening of various antibacterial actives for treatment of asthma and other related symptoms.

### CROSS-REFERENCE TO RELATED APPLICATIONS

This application claims the benefit of U.S. Provisional Application No. 61/141,436, filed Dec. 30, 2008, the content of which is incorporated herein by reference.

### FIELD OF THE INVENTION

The present invention relates to methods of screening actives for the treatment of allergic reactions and providing treatment therefor. In particular, the invention relates to the screening of various antibacterial actives for treatment of asthma and other related symptoms.

### BACKGROUND OF THE INVENTION

The prevalence of allergic disorders in the western world has doubled in the past ten to fifteen years, and is now considered to affect 10-15% of western populations. Allergic asthma is linked with the development of allergic inflammation in the respiratory tract, which leads to airway hyperresponsiveness and corresponding clinical outcome. The present treatment of asthma is inhaled corticosteroid therapy, but there is an ongoing need for a new class of anti-inflammatory treatments.

There is currently no sound and reliable way to determine the proper course of treatment for allergic reactions in individuals, such as asthmatic reactions. Further, currently, treatments for asthmatic reactions are general and not specific to the particular cause of the allergic reaction. For example, certain asthmatic reactions are known to increase the individual's production of IgE. Currently there is no FDA-approved active that may be used to suppress the production of IgE in allergic asthmatics. Recently the benefit of antibiotic agents as part of treatment for certain reactions has been recognized and is growing support for treatment of such diseases. In particular, antibiotic agents such as tigecycline, doxycycline, minocycline and others may be useful in such treatment. However, through the methods and procedures set forth herein, such antibiotics may be administered in a fashion which treats allergic asthmatic responses beyond their known antibacterial effects.

There is currently a need to provide a sufficient method for screening actives for the treatment of specific diseases, and for providing treatment against certain diseases. In addition, there is a further need for a suitable in vitro method for predicting the in vivo properties of such actives.

### SUMMARY OF THE INVENTION

In one embodiment of the present invention there is provided a method of screening actives for treatment in a mammal including the steps of: providing a sample of mammalian peripheral blood mononuclear cells (PBMCs); exposing the mammalian peripheral blood mononuclear cells to an agent to stimulate an allergic response in the mammalian peripheral blood mononuclear cells; treating the stimulated mammalian peripheral blood mononuclear cells with at least one active; and observing the response of the stimulated mammalian peripheral blood mononuclear cells after treatment with the active.

In another aspect of the invention, there is provided a method of screening assays for a disorder including: providing a sample of mammalian peripheral blood mononuclear cells; exposing the mammalian peripheral blood mononuclear cells to an agent to stimulate an allergic response in the mammalian peripheral blood mononuclear cells; treating the stimulated mammalian peripheral blood mononuclear cells with at least one active; and observing the response of the stimulated mammalian peripheral blood mononuclear cells after treatment with the active.

In a further aspect of the invention, there is provided a method of treating a patient including the steps of: screening an active for use in treating the patient including the steps of: extracting from the patient a sample of peripheral blood mononuclear cells; exposing the mammalian peripheral blood mononuclear cells to an agent to stimulate an allergic response in the mammalian peripheral blood mononuclear cells; treating the stimulated mammalian peripheral blood mononuclear cells with at least one active; and observing the treated response of the stimulated mammalian peripheral blood mononuclear cells after treatment with the active; selecting an active which provides a desired treated response; and administering a dosage of the active to the patient.

In another aspect of the invention, there is provided a method of treating an allergic asthmatic through an effective dose of an antibiotic. In a further aspect, the antibiotic may include one or more glycylcyclines, such as tigecycline, doxycycline, minocycline, or combinations thereof.

### BRIEF DESCRIPTION OF THE FIGURES

FIG. 1A depicts the differential levels of cytokines (means and SEM) in culture supernatants of PBMCs from 5 asthmatics at 24 hours p.i. with C. pneumoniae.

FIG. 1B depicts the differential levels of cytokines (means and SEM) in culture supernatants of PBMCs from 10 healthy controls at 24 hours p.i. with C. pneumoniae.

FIG. 2 depicts the IgE production in culture supernatants of PBMC from allergic asthmatics.

FIG. 3 is a graphical depiction of the results of Example 3 set forth herein, demonstrating IgE release by PBMC (1.5×10⁶/ml) from allergic asthmatics (n=11) on day 10 culture+/−C. pneumoniae MOI of 0.1. (A) Logarithmic scale showing levels of IgE (ng/ml) in PBMC culture on day 10 p.i. with Cpn (MOI 0.1) and uninfected (naive) for individual subjects. (B) Data is expressed as mean and SD ng/ml of IgE in cultures with Cpn and uninfected. IgE in culture with Cpn vs uninfected; p=0.008.

FIG. 4 is a graphical depiction of results of Example 3 set forth herein, demonstrating in vitro IgE release by PBMC from study subjects with high and low serum IgE.

FIG. 5 is a graphical depiction of results of Example 3 set forth herein, demonstrating suppression of IgE responses in PBMC cultures day 10 p.i. with C. pneumoniae (Cpn) (MOI=0.1) by doxycycline 1 mg/ml for individual subjects (n=5). FIG. 6 is a graphical depiction of results of Example 3 set forth herein, demonstrating IgE responses to C. pneumoniae (Cpn) and doxycycline in PBMC from serum IgE+allergic asthmatics (n=11) on day 10 of culture.

FIGS. 7A and 7B are graphical depictions of results of Example 3 set forth herein, demonstrating the effect of C. pneumoniae (Cpn) and doxycycline (doxy) on concentration of cytokines A, IL-4 and B, IFN-γ in PBMC from serum IgE+allergic asthmatics (n=11) on day 2 (48 h) of culture.

FIG. 8 is a graphical depiction of results of Example 3 set forth herein, demonstrating differential effect of doxycycline (0.01-1.0 ng/ml) on the concentration of cytokines IL-4 and IFN-γ in PBMC culture from serum IgE+allergic asthmatics (n=11) on day 2 (48 h) p.i. with C. pneumoniae (Cpn) (MOI=0.1).

### DETAILED DESCRIPTION OF THE PREFERRED EMBODIMENTS

Infection with bacteria, such as Chlamydia pneumoniae (C. pneumoniae) is a common cause of allergic reaction, including allergic asthma. Infection with C. pneumoniae is also associated with community-acquired pneumonia (CAP), which likewise is associated with the exacerbation of asthma. It has been found that allergic asthmatics suffer from an increased production of IgE and/or experience a notable cytokine response, which causes an inflammatory response in the asthmatic. In fact, previous studies have shown anti-C. pneumoniae IgE in 86% of culture positive wheezing children, but only in 9% of culture-positive children who were not wheezing. This indicates a role for IgE mediated inflammation that is unique to asthmatics. Through the methods and procedures set forth herein, a suitable treatment for the allergic symptoms may be more easily discovered. Further, through the methods and procedures set forth herein, various combinations of drugs, including antibiotics, may be administered in a fashion which treats allergic asthmatic responses beyond their known antibacterial effects.

The invention provides an in vitro PBMC stimulation model, testing and analyzing a peripheral blood mononuclear cell (also referred to as a “PBMC”). Using the in vitro stimulation model described herein, a simulated response of a patient with allergic asthma is provided. For example, the model may simulate responses of a patient to allergic asthma triggers. Alternatively, the model may simulate responses of a patient through exposure to various treatments. In particular, the preferred embodiment incorporates the use of peripheral blood mononuclear cells (PBMCs) in the stimulation model. The in vitro model set forth herein may be used to predict various in vivo activities, in particular the suppression of IgE production and modulation of the cytokine responses, which will be described in more detail below.

As used herein, the terms “patient”, “subject” and “subject individual” are used interchangeably, and are intended to include the mammal that is the subject of the testing and treatment methods described herein. The term “user” is intended to refer to the individual who is performing the tests, assays, and treatment described herein. Further, the terms “ailment”, “disease” and “condition” are used interchangeably, and refer generally to a condition that causes a responsive affect in the patient. The condition may include, but is not limited to, an allergic response, such as allergic asthma.

In one aspect, the PBMC model of the present invention includes at least one blood sample taken from an allergic asthmatic. PBMCs are isolated from whole blood by any standard method (such as Ficoll gradient). The sample is then treated in such a way so as to provide a desired response. For example, the sample may be stimulated, or mock-infected, by at least one asthmatic trigger. In this fashion, the PBMC sample demonstrates a desired response indicative of an asthmatic patient, and then may then be subjected to various potential treatments to reduce or eliminate the response. The desired response may be any response that results from the exposure or infection with the trigger. Through this inventive model, the user may develop at least one treatment that is effective in reducing and/or eliminating the response triggered by the asthmatic trigger.

For example, in one embodiment, the sample of blood from an asthmatic individual may be stimulated by a trigger such as Chlamydophila pneumoniae (C. pneumoniae), so as to provide a stimulated response. With exposure to C. pneumoniae, one stimulated response may include the upregulation of immunoglobin E (IgE) and cytokine responses. IgE is an antibody, upregulation of which is known to be indicative of an allergic asthma response to a stimulus. The upregulation of IgE has been found to be associated with problematic and potentially detrimental asthmatic activity. Upregulation may indicate that the patient from whom the sample was taken is indeed an allergic asthmatic.

The reduction (or downregulation) of IgE and/or cytokine responses may provide a beneficial treatment to those suffering from asthmatic responses. Treatments that provide such beneficial reduction or suppression of IgE and/or cytokine responses are therefore quite valuable. Determination of such treatment, especially in vitro, may be especially beneficial to avoid potential complications and harmful side effects if administered in vivo.

One potential benefit to using the PBMC stimulation model described herein is to screen various candidate treatments to determine efficacy. Having triggered the stimulated response in the PBMC stimulation model, various candidate treatments may then be assayed and screened to determine their effectiveness in reducing and/or eliminating the stimulated response. As explained above, a particular advantage of using the PBMC stimulation model described herein is that the screening tests may be conducted in vitro, allowing the user to fully screen potential candidates without potential harm to the subject individual. Various treatments may be screened for efficacy, including treatments such as drugs and other active compounds. For example, the PBMC sample, which has been treated with the ailment and thus provides a stimulated response, may be treated with various candidate actives. The response thereto may be monitored and analyzed. In one embodiment, various candidate actives for screening may include antibiotics and drugs from the glycylcycline group, including but not limited to tetracycline, doxycycline, minocycline, tigecycline, and combinations thereof.

In one embodiment, the model described herein may be used to assay blood samples (particularly, PBMC samples) to determine if the patient is an asthmatic. In this embodiment, a sample of blood containing at least one PBMC is taken from a patient and exposed to various triggers, such as C. pneumoniae or any other trigger known to cause a response in asthmatic individuals. A response, such as upregulation of IgE, may indicate that the patient is an allergic asthmatic when exposed to the trigger, and treatment may be provided.

The PBMC stimulation model includes a method for assaying for an allergic response in sample blood, and further may include a method for assaying for various treatments of that allergic response. In some instances, the PBMC stimulation model may be used to assay for a response to a blood-responsive ailment. In this embodiment, first, a subject individual is provided. The subject individual may be a mammal, and may more specifically be a human. In a desired aspect, the subject individual is already known to experience an allergic reaction, such as asthma, from exposure to the ailment for which the sample is being tested. For example, patients who have previously been exposed to a bacteria an experienced a response thereto (such as allergic asthma) may be more likely to exhibit an allergic response when they are later exposed. The patient is preferably known to suffer from the blood-responsive ailment, but need not necessarily be previously known to suffer therefrom.

Once the subject individual is provided, the inventive PBMC method includes the step of taking at least one blood sample from the subject individual, the blood sample including at least one peripheral blood mononuclear cell. In a preferred embodiment, there are provided at least three separate blood samples for the screening methods described herein, each blood sample including at least one PBMC. Peripheral blood mononuclear cells are then extracted from the blood samples, and placed into individual testing sites. In one embodiment, the peripheral blood mononuclear cells may be placed into individual wells of a multi-well plate. Other similar testing sites may be used for the assaying procedures outlined herein.

The peripheral blood cells may then be exposed to an asthmatic stimulus or agent, providing an asthmatic response. This response may be monitored and analyzed by a user. The asthmatic stimulus or agent may include one or more ailments which are known to provide an asthmatic response. For example, the asthmatic stimulus or agent may be a bacterium, such as C. pneumoniae, alternatively, the asthmatic stimulus or agent may include other bacteria. Other stimuli or agents may be similarly assayed using the methods described herein. The asthmatic response is preferably one that may be monitored by a user, such as by providing a physical response of peripheral blood cells when such cells are exposed to the stimulus or agent. One such allergic response includes the upregulation of IgE. In addition, the allergic response may include a promotion of a specific cytokine response, such as increased TH2 response (characterized by TH2 cytokines such as IL-4) which is not balanced by an adequate TH1 response (characterized by TH1 cytokines such as IFN-γ). These responses are understood to be indicative of an allergic response. IL-4 is the prototype IL-4 cytokine; however, additional markers might include expression of CD23 (low-affinity IgE receptor) on PBMC or the presence of soluble CD23 in cell culture, which is not a cytokine. Any other response may be monitored after exposure of the peripheral blood cells to the stimulus or agent. The existence of the allergic response indicates that the subject individual suffers from an allergic response when exposed to the stimulus or agent. If the PBMC does not exhibit the allergic response, it may be determined that the subject individual does not suffer from an allergic response when exposed to the stimulus or agent.

The allergic response monitored may include any one response or a combination of responses. For example, the allergic response may be the upregulation of IgE. Alternatively, the allergic response may be increased TH1 or TH2. Further, the monitored allergic response may be a combination of responses, such as the upregulation of IgE and increased TH1 or TH2. Any other response or responses that are demonstrated by the exposure of peripheral blood cells to a stimulus or agent may be used in the method described herein.

If the PBMC exhibits the asthmatic response being monitored, it may then be assayed for a potential treatment for the stimulus or agent. This may be especially useful shortly after the stimulus has been applied to the PBMC, as it is currently exhibiting the allergic response. A PBMC that exhibits an allergic response is referred to herein as a “responsive PBMC”, and includes any response that is detected. To the responsive PBMC, at least one potential treatment may be applied. The treatment may include the delivery of any active substance, including drugs, antibiotics, vitamins, nutraceuticals, or any medicament desired. The response to the treatment is then monitored and analyzed. In one embodiment, each individual sample of responsive PBMC is exposed to a separate treatment, and each response monitored for a period of time. If desired, more than one sample of responsive PBMC may be treated with the same treatment, to provide more certainty in the assay.

After exposure to the treatment for a period of time, for example, at least 1 hour and up to 14 days (depending on the mechanism and kinetics of the agent), the responsive PBMC sample may be monitored for a response to the treatment (the “treatment response”). For example, when the asthmatic response includes upregulation (or increase) of IgE, the treatment response may include downregulation (or reduction) of IgE. Similarly, when the asthmatic response includes certain cytokine responses, such as increase in TH2 or TH1 responses, the treatment response may include modulation of the cytokine responses. Any reduction in the observed allergic response may be considered a “treatment response”. Further, the level of treatment response may be monitored and analyzed to determine the level of effectiveness of the treatment. For example, if the treatment response may include downregulation of IgE, a 50% downregulation of IgE may indicate that the treatment provides a 50% level of treatment.

In the event that the responsive PBMC sample exhibits one or more treatment responses, the user may consider that the treatment provided was successful in treating the allergic response. It may thus be determined that the treatment provided to the PBMC sample will be effective in treating the subject individual who is experiencing an allergic response to exposure to the stimulus or agent. Further, even if only a percentage of treatment response is observed, it may be determined that the treatment is at least partially effective. This may be useful, for example, when more than one treatments are partially effective and may be combined to fully treat the individual. The PBMC stimulation model may thus be used for drug screening assays and methods of treatment for various diseases.

Any disease or ailment may be screened, and treatment options assayed for. For example, one may wish to screen for treatment of exposure to Chlamydia. A subject individual who experiences allergic response to exposure to Chlamydia is first provided. A sample of blood may be taken from this individual, and from that blood sample, a sample of peripheral blood mononuclear cells may be removed and collected. The PBMC may then be placed into individual wells of a multi-well plate. The PBMC may be exposed to the stimulus or agent (in this case, Chlamydia), and the response may be observed. The PBMC may demonstrate an allergic response as described above, such as upregulation of IgE and/or increased cytokine response. If an allergic response is observed, the user may then determine that the subject individual suffers from allergic response to Chlamydia. The inventive PBMC stimulus model may then be used to assay for a treatment.

The user may then expose the at least one responsive PBMC sample to a treatment, such as an effective dose of tigecycline or any other desired treatment. The PBMC sample that has been exposed to the treatment may then be monitored. Specifically, the level of IgE and/or cytokine may be monitored. In a successful test, indicative of a useful treatment, the PBMC sample may demonstrate reduction in the production of IgE and/or reduced cytokine response. The demonstration of reduction in the production of IgE and/or reduced cytokine response may allow the user to determine that the treatment is effective in treating the allergic response in the subject individual. Thus, the treatment may be administered to the subject individual to treat exposure to the ailment, such as Chlamydia. If more than one treatment is determined to be useful, the treatment applied to the individual may include a combination of treatments. The treatment may be administered via any desired means, including through oral administration, intravenous, intra-arterial, intranasal, suppository, intraperitoneal, intramuscular, intradermal, subcutaneous administration, and combinations thereof. Preferably the treatment is delivered through delivery of an oral medication, including tablets, sprays, or other oral administration.

The inventive PBMC stimulation model may be used in screening drugs for and treatment of many diseases, including but not limited to community-acquired pneumonia, allergic asthma, and atopic dermatitis (also referred to as atopic eczema). So long as the disease produces a response in the peripheral blood mononuclear cells, the PBMC stimulation model may be used to assay for the likelihood that the subject suffers from exposure to the disease. Further the PBMC model may be used to assay for particular a treatment or treatments to the disease. Any treatment may be analyzed by using the PBMC stimulation model described herein. For example, the treatment may include the delivery of an active such as known asthmatic medicaments, such as antibiotics, or any other active desired.

Once the PBMC stimulation method has been completed, and various treatments assayed, the user may then select a treatment to administer to a patient. Desirably the treatment to be administered is one that has been observed and analyzed. Desirably, the treatment selected was one that provided the desired response to the responsive blood sample, including but not limited to the reduction in IgE and/or modulation of cytokine responses. Thus, the user may select a treatment which provides a beneficial effect to the affected blood sample. The treatment may then be applied to the subject individual to treat the disease and/or the symptoms from which the subject individual is suffering.

The PBMC model may be used to screen for various ailments. In this method, a sample of mammalian peripheral blood mononuclear cells from a subject individual is provided. In one embodiment, it is not previously known whether the subject individual suffers from a response to the ailment to be screened. The PBMC sample is then exposed to an ailment, which may stimulate a response related to exposure to the ailment in the peripheral blood mononuclear cells. The response may include, for example, an asthmatic response. The response may then be observed and analyzed by a user. A positive ailment response (for example, observing an allergic response such as upregulation of IgE or other cytokine responses) may indicate that the subject individual suffers from responses due to exposure to the ailment. The user may then treat the stimulated PBMC sample with at least one active to determine the response of the stimulated PBMC sample to the active. The user may then compare the responses of various actives to determine the effectiveness of the various actives, and ultimately may select an active for treatment of the ailment. The ailment may include any blood-responsive ailment, including but not limited to community-acquired pneumonia, allergic asthma, and atopic dermatitis. A non-positive response (i.e., no allergic response observed) may indicate that the subject individual does not suffer from responses due to exposure to the ailment.

Having tested and analyzed at least one potential treatment using the PBMC stimulation method described herein, the user may then select a desired treatment which provides a desired treated response (such as downregulation of IgE, reduction of cytokine response, or combinations thereof). A dosage of the selected active may then be administered to the patient to treat the ailment or the symptoms thereof.

In a desired embodiment, the ailment for which treatments are screened includes allergic asthma resulting from exposure to Chlamydia. The treatments which are screened may include various active agents, including but not limited to tetracycline, doxycycline, minocycline, tigecycline (FDA approved), and others in the glycylcycline group. The active agent may further include one or more antibodies.

It is to be understood that the PBMC model set forth herein may be used to screen for infection with and treatment for any blood-related disease, and is not specifically limited to the diagnosis and/or treatment of allergic asthma. Any disease that generates a response in an individual's blood may be screened using the PBMC model described herein, and likewise treatment for such diseases may also be screened. The present invention is not intended to be limited to one particular ailment and response, such as asthmatic responses, and thus may be useful for screening and treating a plurality of conditions.

In another aspect of the present invention, there is provided a method of treating ailments, such as allergic asthma, with at least one antibiotic. Although various antibacterial effects through the administration of antibiotics are generally known, it has heretofore not been understood the effects that administration of particular combinations of antibiotics may have on treatment of allergic asthma. The present invention seeks to administer a combination of antibiotics as a treatment for other ailments for which such treatment has not been examined.

As set forth herein, there is provided a treatment for allergic asthma and other harmful inflammation in response to an infection through administration of a combination of antibiotics. In one embodiment, the treatment of community-acquired pneumonia includes a macrolide antibiotic in combination with a betalactam antibiotic. Other similar combinations of antibiotics may be administered as desired. The antibiotic may include tetracycline, doxycycline, minocycline, tigecycline (FDA approved), and others in the glycylcycline group. Other antibiotics, which may have anti-inflammatory properties, include macrolides, quinolones, ketolides. Further, the antibiotic may be used alone or in conjunction with other antibiotics, such as β-lactam antibiotic.

Such antibiotics, including macrolide antibiotics, may be useful in treating community-acquired pneumonia. The beneficial effects of such antibiotics, including macroline antibiotics, as well as tetracyclines, go beyond their antibacterial effects against certain ailments, such as M. pneumoniae, L. pneumophila, and C. pneumoniae.

In one aspect, antibiotics, including macrolide antibiotics, may be administered to a patient so as to provide various immunomodulatory effects. Such immunomodulatory effects may include, for example, T-cell, B-cell function and antigen-presenting cell responses, in combination with the downregulation of several inflammatory mediators. The enhancement of protective responses (such as production of chemokines or other molecules that either dampen inflammation and/or eradicate harmful infectious agents) may occur prior to the downregulation of one or more inflammatory mediators. Thus, through the methods and procedures outlined herein, asthmatic responses may be treated quicker than through administration of typical treatments.

The treatments outlined herein may be administered in any desired means. In one embodiment, a patient suffering from allergic asthma, such as that brought about by community-acquired pneumonia, may be treated with an effective dosage of actives such as tetracyclines, or they may be treated with glycylcyclines, such as tigecycline, doxycycline, minocycline, and other related actives. Through an effective dosage of the active, the patient experiences a decreased production of IgE, which is beneficial in treating the allergic response.

The present applicants have discovered the central role that the balance between IL-4 and IFN-γ plays in the regulation of C. pneumoniae mediated IgE production. In particular, it has been found that tetracyclines (including minocycline and doxycycline) have the ability to reduce serum IgE and to provide a steroid sparing effect for allergic asthmatics. Minocycline, along with doxycycline, has also been found to suppress in vitro product of IgE by IL-4/antiCD-40 stimulated PBMC of allergic asthmatics in a dose-dependent fashion. In particular, doxycycline has been shown to suppress immunoglobulin secretion and class switching by activated murine B cells in vitro.

The present invention sets forth a method for concurrently suppressing IL-4 and IgE, while maintaining IFN-γ responses (at least at concentrations<1 μg/mL), thus suggesting that suppression of Th2 lymphocyte functions plays a role in the effects of doxycycline. The present invention may be more fully demonstrated through reference to the Examples set forth below, which are intended to further define the scope of the invention and are not intended to be limiting.

### EXAMPLES

### Example 1

### Determination of Cytokine Levels

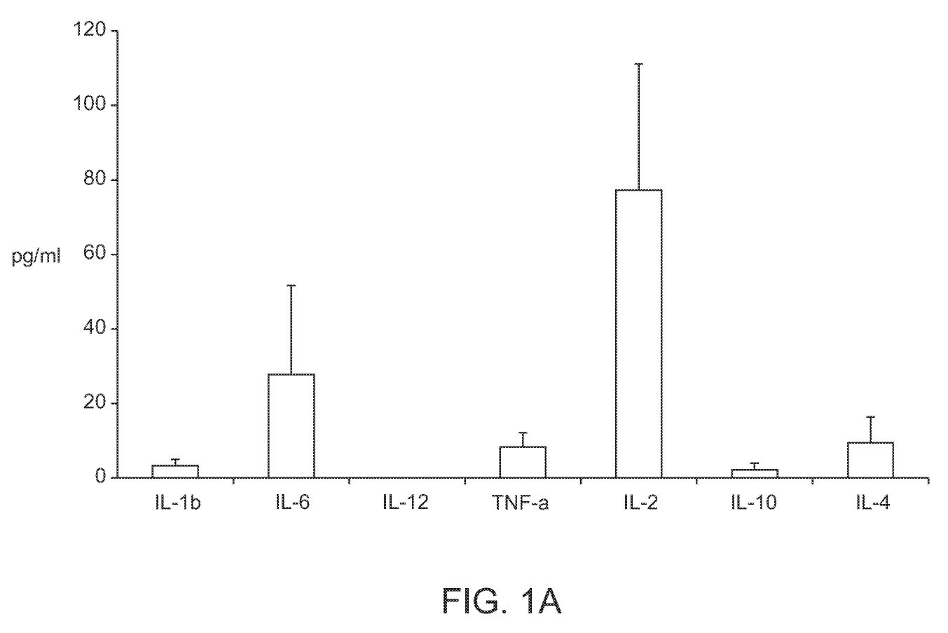

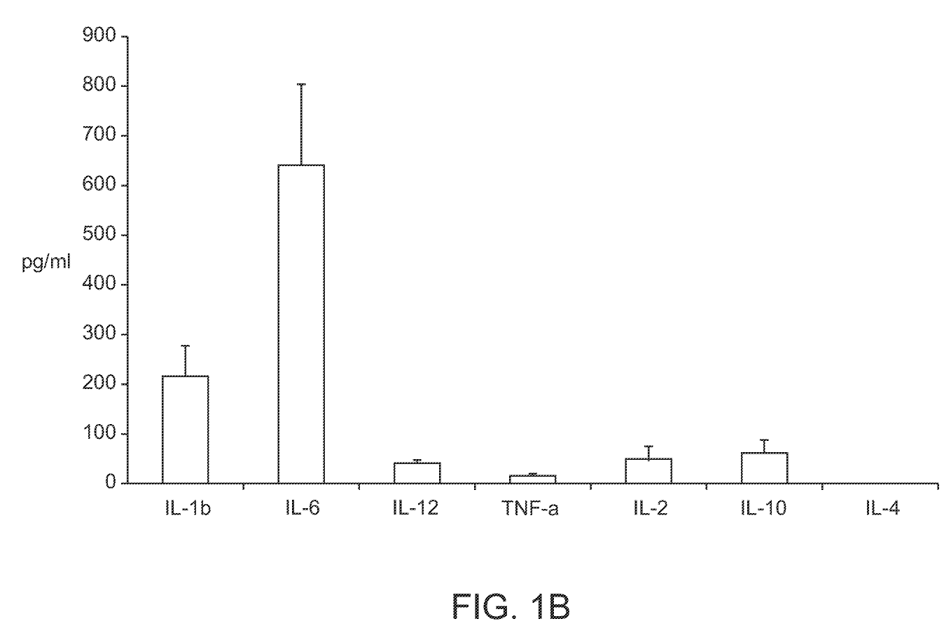

Samples of PBMCs from 5 asthmatics and from 10 healthy controls were taken. For each sample, differential levels of cytokines (means and SEM) in culture supernatants were evaluated at 24 hours post infection with C. pneumoniae TW-183 (MOI 1). The cytokine response due to infection was determined by subtracting the cytokine production from uninfected cells (baseline) from that of infected cells for each subject. The results for the 5 asthmatic samples are set forth in FIG. 1A, and the results for the 10 healthy controls are set forth in FIG. 1B. As can be seen, allergic asthmatics demonstrate an immunomodulatory response to the bacterium. The cell wall of C. pneumoniae lacks the peptidoglycan component, which has been shown to suppress IgE production.

### Example 2

### Assaying Treatments for Cpn

PBMC from allergic asthmatics (serum IgE+) and healthy nonatopic controls (IgE−) were mock-infected or infected with Cpn TW-183 at a MOI=1 for 1 hour. PBMCs were then cultured+/−doxycycline (1 μg/ml) and supernatants were collected on days 2 and 10 post-infection. Th1 (IFN-gamma, IL-12) and Th2 (IL-4) cytokines and levels of total IgE were assayed in supernatants by ELISA.

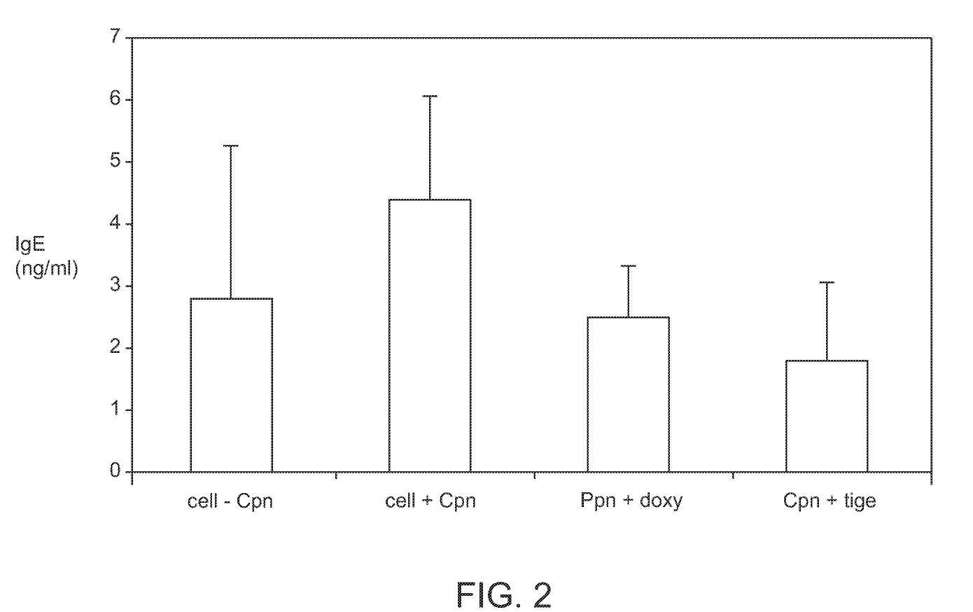

IgE production by PBMC from allergic asthmatics dramatically increased in the presence of Cpn on day 10 (35% p<0.05). IgE increases were accompanied by a Th1 to Th2 switch on day 2. In controls, IgE production did not change. In contrast, Cpn mediated IgE production in PBMCs from asthmatic patients was suppressed (p<0.05) when doxycycline was added to cultures. These results are depicted in FIG. 2. It can thus be concluded that Cpn upregulates IgE production in allergic asthmatics by causing a Th1 to Th2 switch. Tetracyclines, such as tigecycline and doxycycline, show potential as anti-inflammatory drugs in allergic asthmatics.

### Example 3

### Characterization of Immune Simulatory Effects of C. Pneumoniae on PBMC

The immune simulatory effects of C. pneumoniae on PBMC was analyzed in a clinical setting. The clinical characteristics of the enrolled allergic asthmatic patients (n=13) and healthy controls are shown in Table 1 below. All asthmatics were serum IgE positive at the time the blood specimens were obtained with a mean serum IgE of 614±512 IU/ml. The baseline characteristics of healthy controls were comparable to those of asthmatics except for IgE, which was significantly lower. Both the rate of C. pneumoniae seropositivity and the median titer as determined by MIF were not statistically different between controls and asthmatics with about half of subjects in each group showing evidence of previous exposure.

TABLE 1
Study subjects - clinical characteristics

| | Allergic | Healthy control |
|---|---|---|
| | asthmatics | subjects |
| N | 13 | 12 |
| Age (years) | 46 ± 17 | 36 ± 12 |
| Sex ratio (F/M) ^{a} | 15/4 | 7/5 |
| AQLQ score | 150 ± 44 | — |
| FEV1 (% pred) | 70 ± 17 | — |
| FVC (% pred) | 79 ± 17 | — |
| eNO (ppb) ^{b} | 38 ± 41 | — |
| Serum IgE (IU/ml) | 614 ± 512 * | 23 ± 25 * |
| Cpn serology | 54 | 50 |
| (% positive) ^{c} | | |
| Median MIF titer | 1:32 | 1:32 |
| Systemic cortico- | 31 | — |
| steroid therapy (%) | | |

^{a} F/M = female/male
^{b} eNO = exhaled nitric oxide
^{c} C. pneumoniae serology by micro immunofluorescence (MIF) titer ≧ 16
* p = 0.001

Allergic asthmatics and healthy controls were consecutively enrolled in the outpatient clinic at SUNY Downstate Medical Center from July 2006 to October 2007. Asthmatics had to have stable disease, without current or recent symptoms of asthma exacerbation or acute respiratory infection (within the past 3 months). Controls were without history of atopic diseases or asthma and without current or recent symptoms suggestive of acute respiratory infection. Asthma severity was evaluated in accordance with National institutes of Health guidelines. Serum IgE levels were determined by fluroenzymeimmunoassay (UniCAP, Pharmacia, Uppsala, Sweden).

### Preparation of Materials

Specific anti-C. pneumoniae serum IgG antibodies were detected, using a commercial microimmunofluorescence (MIF) kit (Labsystems, Helsinki, Finland). Each kit contains positive antigen and negative controls. Sera were titrated in twofold serial dilutions, the positivity criterion of C. pneumoniae-specific serum antibodies was IgG≧1:16. Antibody titers were read by two persons in a blinded fashion.

Peripheral blood (15 mL total) was obtained from patients and controls in ethylenediaminotetraacetic (EDTA) tubes. PBMC were isolated from whole blood on Ficoll-Paque (GE Healthcare, Sweden) and then washed in RPMI 1640 with 10% fetal bovine serum (FBS), and resuspended in complete RPMI 1640 (c-RPMI). C-RPMI contained RPMI-1640 Medium HEPES Modification supplemented by L-glutamine 5 mM and 10% FBS. Cells were counted and cell viability evaluated with trypan blue dye using a hemocytometer. PBMC (1.5×10⁶/mL) were cultured in duplicate in 24 flat bottom well plates (1 ml/well) for up to 12 days in absolute humidification and 5% CO₂. Cell viability at 0, 48 and 240 hours was >95%. Following a 2 h incubation to allow adherance PBMC cultures were infected or stimulated in the presence or absence of doxycycline. Doxycycline was reconstituted in a 1× sterile Dulbecco's Phosphate Buffered Saline (PBS).

C. pneumoniae TW-183 (ATCC 53592) was be obtained from the ATTC (Manassas, Va.). C. pneumoniae culture aliquots were frozen. Briefly, HEp-2 cell (ATCC LCL-23) monolayers were inoculated with C. pneumoniae and centrifuged at 1,700×g for 1 h and then incubated for 1 h at 35° C. Fresh overlay medium, Iscove's modified Dulbecco's medium (GIBCO) containing 1 μg/ml cycloheximide and 10% fetal calf serum, was added, and plates were incubated for 72 h at 35° C. Cell layers were scraped off the plates, vortexed with glass beads, pooled, and transferred to fresh HEp-2 cell monolayers. Several passages were required to reach the concentration and volume needed for assays. Suspensions of C. pneumoniae elementary bodies were purified by layering HEp-2 cell lysates over 23% sucrose in Oakridge tubes and centrifuging at 5856 g for 1 h. The band containing the purified elementary bodies was suspended in sucrose phosphate buffer. Aliquots were frozen at −80° C. and, following thawing, the titer of the aliquots was determined (approximately 1×10⁷ IFU/mL). For determining, the infectious titers plates were fixed at 72 h post infection (p.i.), stained with an anti-lipopolysaccharide (i.e., genus antigen) monoclonal antibody, and inclusions per well were counted.

PBMC were infected by adding purified EB at a multiplicity of infection (MOI) of 0.01-5 for 1 hour, followed by further incubation in the presence or absence of doxycycline, at 0.01, 0.1, 1.0 μg/mL, for 2 and 10 days p.i. at 37.0° C. in complete medium in a humidified 5% CO₂ atmosphere. The time points 2 d (for cytokines) and 10 d p.i. (for IgE) were determined by kinetic studies for optimization of the assay, which revealed peak concentrations and clear distinctive profiles for the respective outcome variables at these time points. The most effective MOI for the induction of lymphocyte cytokines and IgE was MOI=0.1, which was used in all experiments. Adherent cells were stained with monoclonal genus specific antibodies to confirm and quantify infection with C. pneumoniae at 72 h post infection. Identical volumes of inactivated C. pneumoniae were added in some experiments. C. pneumoniae was inactivated by incubation in a 56° C. water bath for 30 minutes. Aliquots were then added to HEp-2 cultures as described for titration of live C. pneumoniae; no inclusions were observed following inoculation with the heat-treated bacteria. Mock-infection was carried out by adding equal volumes of HEp-2 cell cultures not containing any bacteria processed the same way as the purified C. pneumoniae.

### Results of Experiments

### C. Pneumoniae Enhances in Vitro IgE Synthesis by PBMC of Allergic Asthmatics.

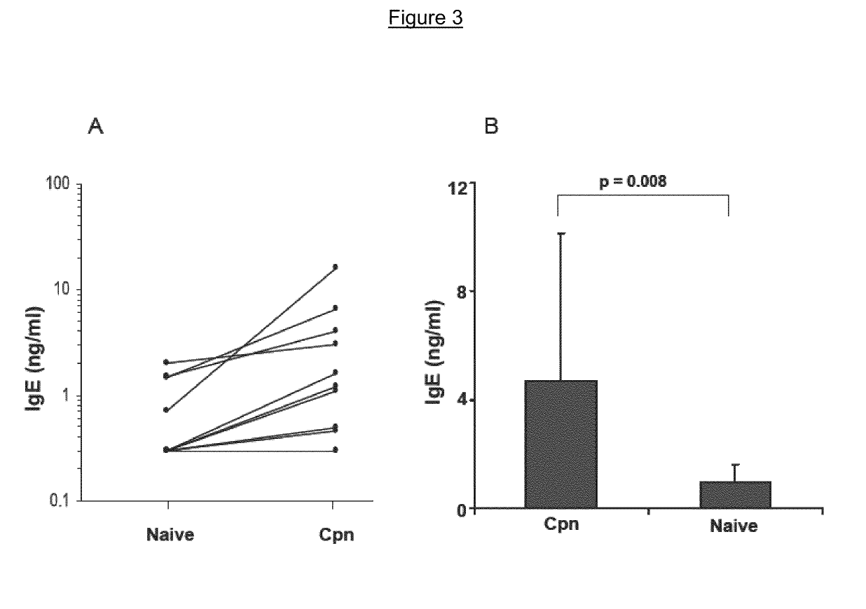

In pilot experiments using a C. pneumoniae multiplicity of infection (MOI) of 0.01-10, maximal IgE responses in PBMC culture were found with a MOI of 0.1-1 on day 10 p.i., which were comparable to those induced by IL-4, anti-CD40 or IL-4+anti-CD40. When PBMC (1.5×10⁶) from allergic asthmatics were cultured in the presence or absence of C. pneumoniae (MOI=0.1), augmentation of IgE responses by C. pneumoniae were observed on day 10 (4.7±5.4 versus 0.9±0.7 ng/ml, p=0.008) (See FIG. 3). Culturing PBMC with a MOI of 1.0 or heat inactivated C. pneumoniae did not result in statistically significant IgE changes. IgE levels in cell culture for nonasthmatic controls were found to be below the IgE ELISA detection limit (0.5 ng/ml) for all conditions.

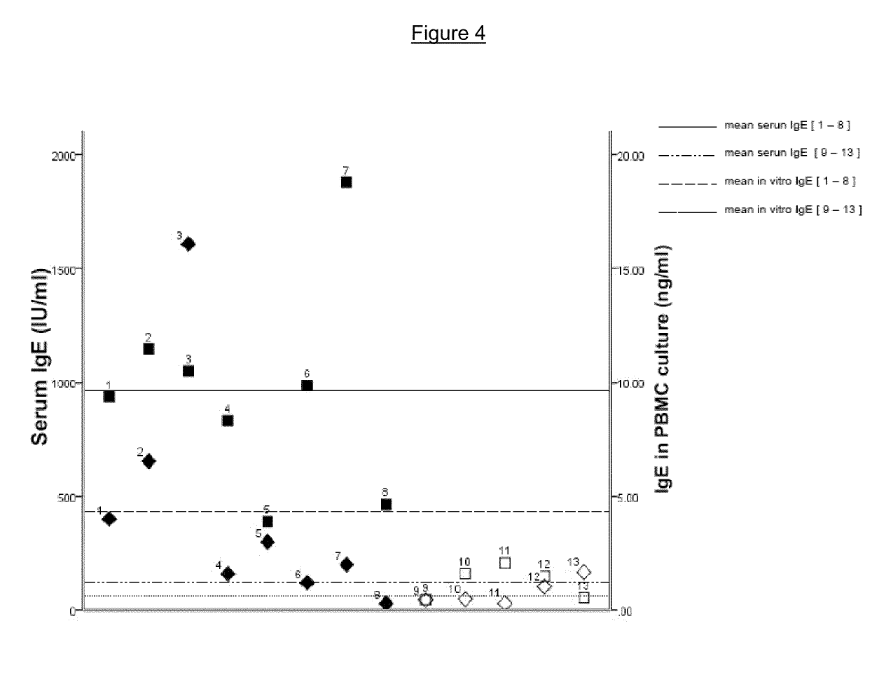

In addition, there was increased IgE release in PBMC culture with C. pneumoniae for study subjects with high serum IgE levels (≧200 IU/ml). In contrast, IgE release in PBMC culture for study subjects with serum IgE of less than 200 IU/ml was negligible (See FIG. 4). In FIG. 4, the data is shown as pairs of serum and in vitro IgE levels for each subject as indicated by subject number; serum IgE (IU/ml) (squares) and in vitro IgE (ng/ml) in PBMC culture day 10 p.i. with C. pneumoniae MOI=0.1 (diamonds) for individual patients. Subjects were grouped as those with high serum IgE of (≧200 IU/ml): 1-8 (filled symbols) on the left; and subjects with serum IgE less than 200 IU/ml: 9-13 (open symbols) on the right. Means of serum IgE (IU/ml) are shown as a solid line for subjects 1-8, dashes with dots for 9-13, and in vitro IgE (ng/ml) is shown as dashed line for subjects 1-8 and small dots for 9-13. In vitro and in vivo IgE level pairs for individual subjects are also shown in FIG. 4. It was found that no allergic asthmatic without a positive C. pneumoniae IgG titer secreted IgE in PBMC culture with C. pneumoniae.

### C. Pneumoniae Augments Production of IL-4 by PBMC of Allergic Asthmatics.

In order to characterize the contribution of different T lymphocyte subpopulations to in vitro IgE production, cytokine concentration in infected and uninfected PBMC cultures were analyzed and determined. IL-4 and IFN-γ were chosen as representative cytokines for Th2 and Th1 responses, respectively.

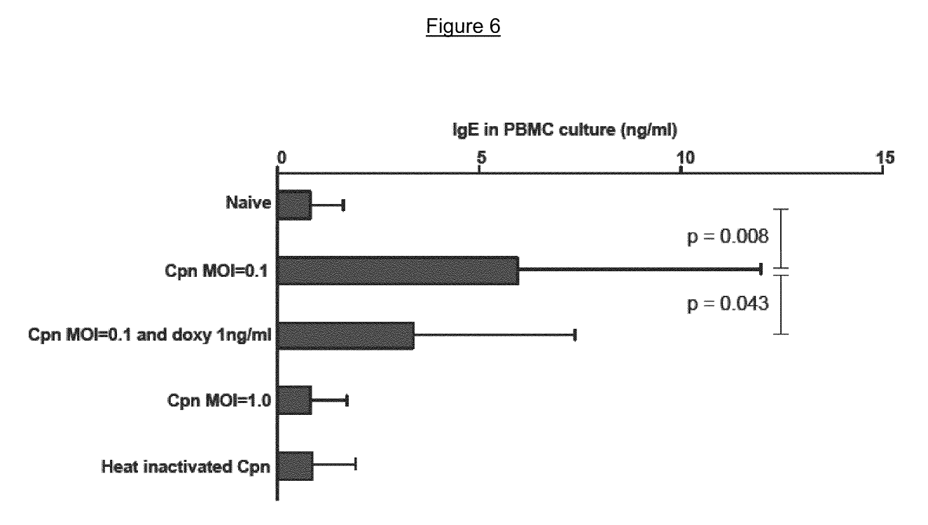

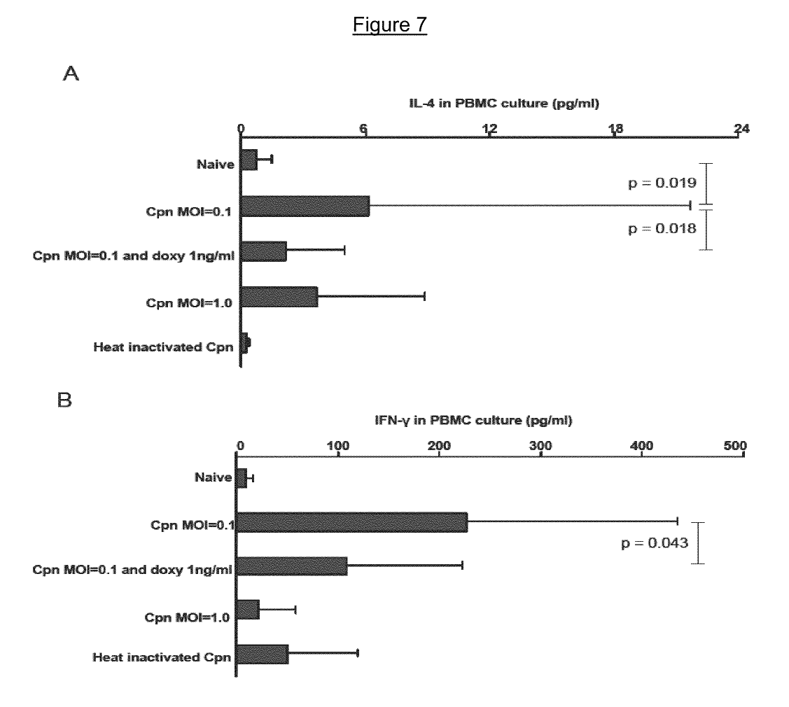

In allergic asthmatics, PBMC release of IL-4 was markedly increased in response to C. pneumoniae (MOI=0.1) infection (mean 6.2±15.5 pg/ml) versus uninfected (mean 0.7±0.8 pg/ml, p=0.019), while the IL-4 concentration in PBMC cultures from controls were low in uninfected (0.1±0.3 pg/ml) and infected cells (0.2±0.3 pg/ml, p=NS). PBMC culture mean levels of IFN-γ were higher in controls (uninfected 12.5±8.3 pg/ml vs infected 498±552 pg/ml; p=NS) than in asthmatics (uninfected 9.4±6.6 pg/ml vs infected 226±208, p=NS). The IL-4 and IFN-γ responses of PBMC from allergic asthmatics to different stimuli are shown in FIGS. 7A and 7B. In FIGS. 7A and 7B, the data are expressed as means±SD of IL-4 (pg/ml) in cultures with the following conditions: uninfected cells (Naïve); infected with Cpn in multiplicity of infection (MOI) of 0.1; Cpn MOI=0.1 and doxycycline 1 mg/ml; Cpn MOI=1.0 and heat inactivated Cpn.

The C. pneumoniae-medicated in vitro increase in IgE was correlated with C. pneumoniae induced IL-4 (CC=0.979; p<0.001), but not with IFN-γ production. In addition, in vitro IL-4 production was correlated with in vitro IgE production in C. pneumoniae infected PBMC (CC=0.979; p<0.001). Only allergic asthmatics with positive C. pneumoniae IgG titers had increases of in vitro IL-4 production when PBMC cultures were infected with C. pneumoniae.

### Doxycycline Decreases in Vitro IgE Release by PBMC and Production IL-4.

The ability of doxycycline and minocycline to decrease in vitro IgE responses in cultures of PBMC stimulated by C. pneumoniae infected PBMC was determined.

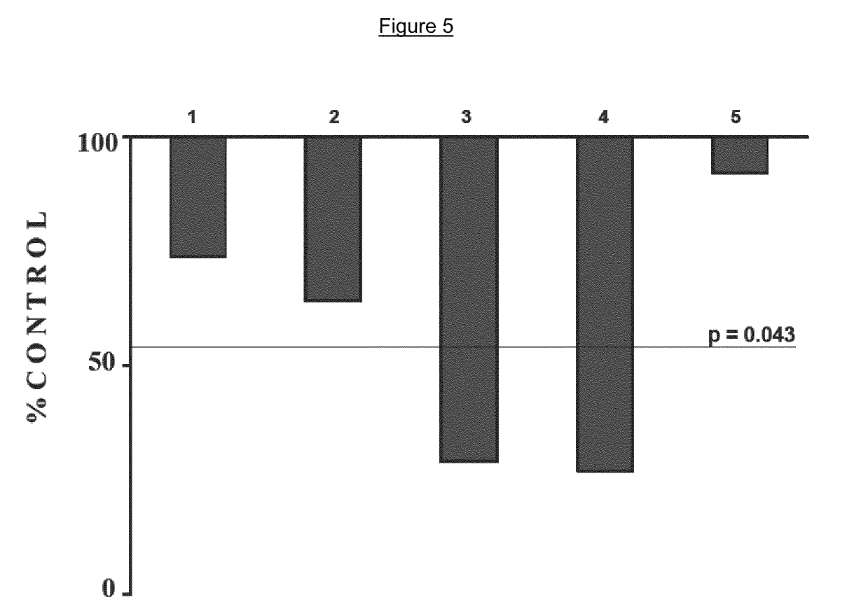

Doxycycline at a concentration of 1 μg/ml was added to PBMC cultures after one hour incubation with C. pneumoniae MOI of 0.1. Addition of doxycycline in culture suppressed IgE responses by an average of 42% (3.4±3.9 versus 5.9±6.0 ng/ml, p=0.043) as shown in FIGS. 5 and 6. In FIG. 5, the data is expressed as percent control (PBMC day 10 p.i.); the mean change was 48% decrease; p=0.043. In FIG. 6, the data is expressed as mean IgE±SD (ng/ml) in cultures with the following conditions: uninfected cells (Naïve); Cpn MOI=0.1; Cpn MOI=0.1 and doxycycline 1 mg/ml; Cpn MOI of 1.0 and heat inactivated Cpn.

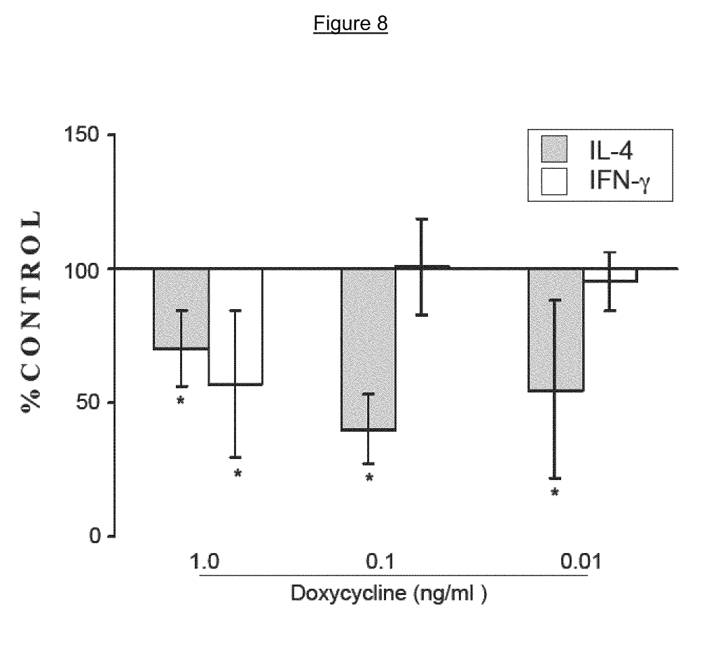

To determine the effect of doxycycline on cytokine release by PBMC, doxycycline (0.01-1.0 ng/ml) was added to culture following infection with C. pneumoniae (MOI=0.1). In addition to suppression of IgE release by PBMC cultures, addition of doxycycline 1 ng/ml markedly decreased both IL-4 and IFN-γ levels with mean % decrease of 30±14 (p=0.018) and 43±27 (p=0.043), respectively. Addition of lower concentrations of doxycycline 0.01 μg/ml and 0.1 μg/ml markedly decreased levels of IL-4 (mean % change of −45±33, p=0.018, and −60±13, p<0.05) but not IFN-γ (mean % change 5%±11, p=NS, and −1%±18, p=NS) (See FIG. 8). In FIG. 8, the data is expressed as a percent control (PBMC day 10 p.i.), 100% is the mean highest response to Cpn; *P<0.05 for doxycycline treated cultures versus no doxycycline. Interestingly, the effects of doxycycline on IL-4 suppression were observed even at 0.01 μg/ml, which indicates a subinhibitory concentration for C. pneumoniae.

This Example demonstrates an in vitro anti-inflammatory effect that can be observed over a wide range of concentrations down to levels below the reported MIC of doxycycline for C. pneumoniae. This suggests that tetracyclines suppress C. pneumoniae mediated IgE responses and cytokine production in the presence of C. pneumonia infection. This is at least in part mediated by selective suppressing IL-4, which, as described above, plays an important part in producing and maintaining inflammation in asthma. This demonstrates that PBMC of patients with allergic asthma contain activated B-cells that continue to secrete IgE in vitro, and are responsive to infection with C. pneumoniae. This response is correlated with IL-4 production, but not IFN-γ production, indicating a role of Th2 lymphocytes. This provides an effective and useful method of not only screening for potential issues, but for treating as well.

What is claimed is:

1. A method of screening actives for treatment in a mammal comprising the steps of:
a. Providing a sample of mammalian peripheral blood mononuclear cells;
b. Exposing said mammalian peripheral blood mononuclear cells to an agent to stimulate an allergic response in said mammalian peripheral blood mononuclear cells;
c. Treating said stimulated mammalian peripheral blood mononuclear cells with at least one active; and
d. Observing the response of said stimulated mammalian peripheral blood mononuclear cells after treatment with said active.

2. The method of claim 1, wherein said mammal is a human.

3. The method of claim 1, wherein said agent is chlamydophila pneumoniae.

4. The method of claim 1, wherein said allergic response is the upregulation of IgE.

5. The method of claim 1, wherein said allergic response comprises cytokine response.

6. The method of claim 1, wherein said active comprises medications for the treatment of asthma.

7. The method of claim 1, wherein said active comprises an antibiotic.

8. The method of claim 1, wherein said active comprises a medication selected from the group consisting of tetracycline, doxycycline, minocycline, tigecycline, and combinations thereof.

9. The method of claim 4, wherein said step of observing comprises observing the reduction in IgE production.

10. The method of claim 5, wherein said step of observing comprises observing the reduction in cytokine response.

11. The method of claim 1, further comprising the step of selecting an active after said step of observing the response.

12. The method of claim 1, wherein said method is performed ex vivo.

13. A method of screening assays for a disorder comprising:
a. Providing a sample of mammalian peripheral blood mononuclear cells;
b. Exposing said mammalian peripheral blood mononuclear cells to an agent to stimulate an allergic response in said mammalian peripheral blood mononuclear cells;
c. Treating said stimulated mammalian peripheral blood mononuclear cells with at least one active; and
d. Observing the response of said stimulated mammalian peripheral blood mononuclear cells after treatment with said active.

14. The method of claim 13, wherein said disorder is selected from the group consisting of community-acquired pneumonia, allergic asthma, and atopic dermatitis.

15. A method of treating a patient comprising the steps of:
a. Screening an active for use in treating said patient comprising the steps of:
i. Extracting from said patient a sample of peripheral blood mononuclear cells;
ii. Exposing said mammalian peripheral blood mononuclear cells to an agent to stimulate an allergic response in said mammalian peripheral blood mononuclear cells;
iii. Treating said stimulated mammalian peripheral blood mononuclear cells with at least one active; and
iv. Observing the treated response of said stimulated mammalian peripheral blood mononuclear cells after treatment with said active;
b. Selecting an active which provides a desired treated response; and
c. Administering a dosage of said active to said patient.

16. The method of claim 15, wherein said mammal is a human.

17. The method of claim 15, wherein said agent is chlamydophila pneumoniae.

18. The method of claim 15, wherein said allergic response is the upregulation of IgE.

19. The method of claim 15, wherein said allergic response comprises cytokine response.

20. The method of claim 15, wherein said active comprises medications for the treatment of asthma.

21. The method of claim 15, wherein said active comprises an antibiotic.

22. The method of claim 15, wherein said active comprises a medication selected from the group consisting of tetracycline, doxycycline, minocycline, tigecycline, and combinations thereof.

23. The method of claim 18, wherein said step of observing comprises observing the reduction in IgE production.

24. The method of claim 19, wherein said step of observing comprises observing the reduction in cytokine response.

25. The method of claim 15, further comprising the step of selecting an active after said step of observing the treated response.

26. The method of claim 15, wherein said method is performed ex vivo.
